(19) United States
(12) Patent Application Publication
(10) Pub. No.: US 2026/0088181 A1
(43) Pub. Date: Mar. 26, 2026

(54) METHOD FOR CALCULATING BLOOD FLOW AND SHEAR STRESS OF AORTA HAVING A BICUSPID AORTIC VALVE, READABLE MEDIUM STORING THE SAME AND MEDICAL DEVICE USING THE SAME

(71) Applicant: University of Seoul Industry Cooperation Foundation, Seoul (KR)

(72) Inventor: Do Yeol AHN, Seoul (KR)

(73) Assignee: University of Seoul Industry Cooperation Foundation, Seoul (KR)

(21) Appl. No.: 18/936,705

(22) Filed: Nov. 4, 2024

(30) Foreign Application Priority Data
Sep. 20, 2024 (KR) 10-2024-0127457

(51) Int. Cl. G16H 50/30; G06F 17/11

(52) U.S. Cl. CPC G16H 50/30; G06F 17/11

(57) ABSTRACT

Disclosed are a method for calculating blood flow and shear stress of aorta having a bicuspid aortic valve, readable medium storing the method and a medical device using the method. A method for calculating blood flow and shear stress of aorta having a bicuspid aortic valve, which is performed by an electronic device, the method comprises receiving characteristic values of a bicuspid aortic valve, and calculating blood flow per unit area and shear stress applied to the aorta in a bipolar coordinate system of governing equation.

### CROSS REFERENCE TO RELATED APPLICATION

This application claims priority from and the benefit of Korean Patent Applications No. 10-2024-0127457, filed on Sep. 20, 2024, which is hereby incorporated by reference for all purposes as if fully set forth herein.

### BACKGROUND OF THE INVENTION

### Field of the Invention

The present invention relates to a method for calculating blood flow and shear stress of aorta having a bicuspid aortic valve, readable medium storing the method and medical device using the method.

### Discussion of the Background

In the cardiovascular system, there are valves to prevent the backflow of blood. These valves are membrane-shaped structures, and the tricuspid aortic valve (TAV) that normal people have has a membrane that is divided into three leaflets in a “Y” shape in the cross section of the blood vessel. However, some people have a congenital malformation, a bicuspid aortic valve (BAV) that is divided into two leaflets in a “1” shape in the blood vessel, as shown in FIG. 1.

Bicuspid aortic valve (BAV) is the most common congenital heart defect, affecting approximately 1-2% of the population. It is caused by the presence of only two leaflets in the aortic valve instead of three, which can lead to potential cardiovascular problems. The main problem is valve dysfunction, which can lead to stenosis (stricture) or regurgitation (leakage). Patients with BAV are also at higher risk for aortic dilatation, which can lead to life-threatening aortic dissection, and an increased risk of infective endocarditis. These risks require careful monitoring and timely intervention in patients with BAV because of the altered valve mechanics and increased aortic wall stress.

To this end, the conventional finite element method (F.E.M.) is used to calculate the blood flow in the aorta with a bicuspid aortic valve and the shear stress applied to the aortic vessel inner wall using a simulation based on the Navier-Stokes equations. However, this method has the problem of taking a lot of time.

### SUMMARY OF THE INVENTION

Therefore, the present invention provides a method for calculating blood flow and shear stress of an aorta having a bicuspid aortic valve, which can reduce the calculation time.

The present invention also provides a a medical device using the method for calculating blood flow and shear stress of an aorta having such a bicuspid aortic valve.

A method for calculating blood flow and shear stress of aorta having a bicuspid aortic valve, which is performed by an electronic device, the method comprises receiving characteristic values of a bicuspid aortic valve, and calculating blood flow per unit area and shear stress applied to the aorta in a bipolar coordinate system of governing equation.

For example, the characteristic values of a bicuspid aortic valve are interpole distance (2a) in the bipolar coordinate system and the corresponding ξ_{*} value at the valve edge.

For example, the governing equation is expressed as

$$\frac{\partial P}{\partial z} = μ ( \frac{{\partial }^{2} u}{\partial {x}^{2}} + \frac{{\partial }^{2} u}{\partial {y}^{2}} )$$

wherein P is the pressure, u(x,y) is the velocity in the z-direction, μ is the viscosity of the blood, and x and y represent each axis of the Cartesian coordinate system, respectively.

For example, a solution of the governing equation in the bipolar coordinate system is obtained by a boundary condition expressed as

$${u}_{0} ( {ξ}_{*} \text{?} η ) = 0 , {u}_{0} ( 2 π - {ξ}_{*} ) = 0$$

$$\text{?} \text{indicates text missing or illegible when filed}$$

For example, a steady state solution obtained by the boundary condition is expressed as

$${u}_{0} ( ξ \text{?} η ) = - \frac{k \text{?}}{2 μ} \frac{sin ( ξ - {ξ}_{* j} )}{sin ( {ξ}_{* j} ) ( cosh η - cos \text{?} )} ,$$

$$wherein$$

$${ξ}_{* j} = {ξ}_{*} for {ξ}_{*} \leq ξ \leq π and {ξ}_{* j} = 2 π - {ξ}_{*} for π \leq ξ \leq 2 π - {ξ}_{*} .$$

$$\text{?} \text{indicates text missing or illegible when filed}$$

For example, a solution involving time t due to the contraction of the heart is approximated by multiplying the steady-state solution by the value of e^{iωt}.

For example, wherein a blood flow Q obtained by integrating the steady-state equation is expressed as

$$Q = \frac{k}{4 μ} [ \text{} {a}^{4} cot {ξ}_{*} ( \frac{4}{3} + \frac{4}{3} {cot}^{2} {ξ}_{*} - 4 {csc}^{2} {ξ}_{*} ) + {a}^{4} {csc}^{4} {ξ}_{*} ( - {ξ}_{*} - \frac{2}{3} sin 2 {ξ}_{*} - \frac{1}{16} sin 4 {ξ}_{*} ) ]$$

For example, a shear stress τ(ξ,η) applied to the aorta obtained from the above steady-state equation is expressed as

$$\begin{matrix}τ = - μ \frac{cosh η - cos ξ}{a} \frac{\partial u ( ξ \text{?} η )}{\partial ξ} \\ = \frac{ka}{2 sin \text{?}} [ cos ( ξ - {ξ}_{* j} ) - \frac{sin ( ξ - {ξ}_{* j} ) sin ξ}{cosh η - cos ξ} ] ,\end{matrix}$$

$$\text{?} \text{indicates text missing or illegible when filed}$$

For example, a shear stress at the end (ξ*j) of a bicuspid aortic valve is expressed as

$$τ {\vert}_{{ξ}_{* j}} = \frac{ka}{2 sin {ξ}_{* j}}$$

A computer-readable recording medium for performing the method for calculating blood flow and shear stress of an aorta having a bicuspid aortic valve according an exemplary invention performs a method described any one of above.

A medical device using the method for calculating blood flow and shear stress of an aorta having a bicuspid aortic valve according to any one of above, comprises an input unit, a calculation unit and an output unit. The input unit receives characteristic values of a bicuspid aortic valve. The calculation unit calculates blood flow per unit area and shear stress applied to an aorta in a bipolar coordinate system of a governing equation using the characteristic values received from the input unit. The output unit outputs the result calculated from the association unit.

For example, the characteristic values may be received from a user.

On the other hand, the characteristic values may be received from a measurement unit.

As described above, according to the method for calculating blood flow and shear stress of the aorta by the present invention, compared to the conventional method of using a simulation by the Navier-Stokes equation using the finite element method (F.E.M.), the bicuspid aortic valve is approximated by a bipolar coordinate system having a similar shape, and the directly obtained solution is used, thereby drastically reducing the speed of calculation and enabling rapid diagnosis.

### BRIEF DESCRIPTION OF THE DRAWINGS

FIG. 1 is a schematic diagram illustrating a bicuspid aortic valve.

FIG. 2 is a flowchart illustrating a method for calculating blood flow and shear stress of an aorta having a bicuspid aortic valve.

FIG. 3 illustrates a bipolar coordinate system for approximating the bicuspid aortic valve illustrated in FIG. 1.

FIG. 4A illustrates the steady flow through the bipolar-shaped orifice, depicting the wall boundaries at positions ξ_{*}=2π/3, for bicuspid aortic valve.

FIG. 4B illustrates the steady flow through the bipolar-shaped orifice, depicting the wall boundaries at positions ξ_{*}=3π/4, for bicuspid aortic valve.

FIG. 4C illustrates the steady flow through the bipolar-shaped orifice, depicting the wall boundaries at positions ξ_{*}=4π/5, for bicuspid aortic valve.

FIG. 4D illustrates the steady flow through the bipolar-shaped orifice, depicting the wall boundaries at positions ξ_{*}=5π/6, for bicuspid aortic valve.

FIG. 5A presents comparative velocity profiles at the aorta entrance for BAV.

FIG. 5B presents comparative velocity profiles at the aorta entrance for TAV.

FIG. 5C presents comparative velocity profiles at the aorta entrance for a combined profile showing BAV (blue) and TAV (red) at the center.

FIG. 6A illustrates the normalized shear stress distribution across the bipolar-shaped orifice, highlighting the wall boundaries at positions ξ_{*}=2π/3.

FIG. 6B illustrates the normalized shear stress distribution across the bipolar-shaped orifice, highlighting the wall boundaries at positions ξ_{*}=3π/4.

FIG. 6C illustrates the normalized shear stress distribution across the bipolar-shaped orifice, highlighting the wall boundaries at positions ξ_{*}=4π/5.

FIG. 6D illustrates the normalized shear stress distribution across the bipolar-shaped orifice, highlighting the wall boundaries at positions ξ_{*}=5π/6.

FIG. 7 illustrates the wall shear stress (WSS) of the bicuspid aortic valve (BAV) normalized by the WSS of the tricuspid aortic valve (TAV).

FIG. 8 is a block diagram illustrating a medical device according to an exemplary embodiment of the present invention.

FIG. 9 is a block diagram illustrating a medical device according to another exemplary embodiment of the present invention.

### DETAILED DESCRIPTION OF THE ILLUSTRATED EMBODIMENTS

The present invention is described more fully hereinafter with reference to the accompanying drawings, in which example embodiments of the present invention are shown. The present invention may, however, be embodied in many different forms and should not be construed as limited to the example embodiments set forth herein. Rather, these example embodiments are provided so that this disclosure will be thorough and complete, and will fully convey the scope of the present invention to those skilled in the art. In the drawings, the sizes and relative sizes of layers and regions may be exaggerated for clarity.

It will be understood that, although the terms first, second, etc. may be used herein to describe various elements, these elements should not be limited by these terms. These terms are only used to distinguish one element from another element. For example, a first element discussed below could be termed a second element, and similarly, a second element may also be termed a first element, without departing from the teachings of the present invention.

The terminology used herein is for the purpose of describing particular example embodiments only and is not intended to be limiting of the present invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, components, and/or groups thereof but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs.

It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The present invention relates to a functional sheet manufactured through a powder spray method capable of enhancing safety and function due to not using an organic solvent which is harmful to the human body, and a method for manufacturing the functional sheet.

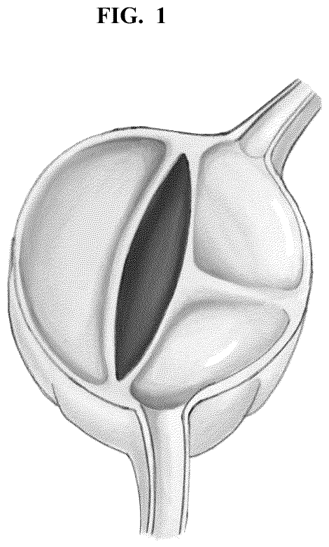

Hereinafter, referring to FIG. 1, a method of manufacturing a functional sheet through a powder spray method according to an embodiment of the present invention will be described in more detail.

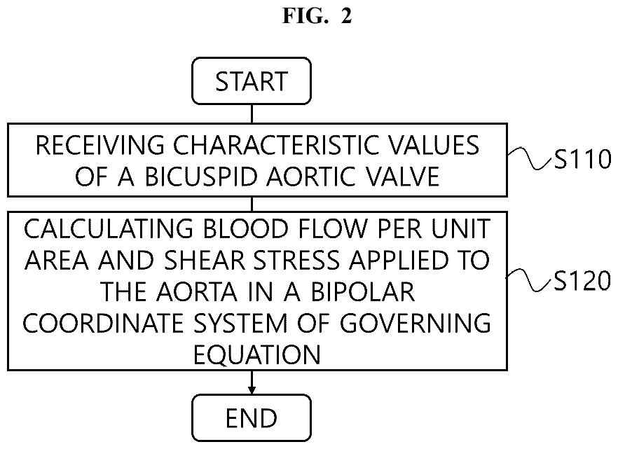

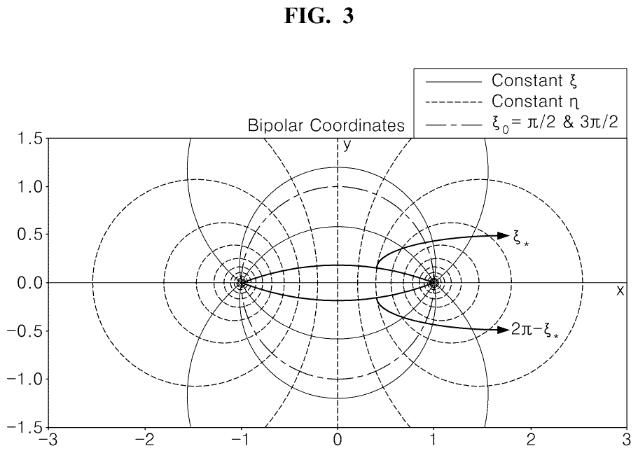

FIG. 2 is a flowchart illustrating a method for calculating blood flow and shear stress of an aorta having a bicuspid aortic valve.

Referring to FIG. 2, the method for calculating blood flow and shear stress of the aorta according to an exemplary embodiment of the present invention includes a step (S110) in which an electronic calculator receives a characteristic value of a bicuspid aortic valve, and a step (S120) in which the characteristic value of the bicuspid aortic valve is used to calculate the blood flow per unit area and the shear stress applied to the aorta in the bipolar coordinate system of the governing equation.

As an example, the characteristic value of the bicuspid aortic valve may be a distance between poles (2a) and a value corresponding to the valve end in the bipolar coordinate system.

Hereinafter, it will be described in more detail.

When the pressure gradient k acts in the axial direction of the tube of the bipolar cross section, the governing equation for the steady flow is expressed as the following Expression 1.

$$\frac{\partial P}{\partial z} = μ ( \frac{{\partial }^{2} u}{\partial {x}^{2}} + \frac{{\partial }^{2} u}{\partial {y}^{2}} ) , \qquad [ Equation 1 ]$$

In this equation, P is the pressure, u(x,y) is the velocity in the z-direction, and μ is the viscosity of the blood. Also, here, x and y represent each axis of the Cartesian coordinate system, respectively.

It is shown that the transverse components of the velocity are identically zero.

For the problem we are addressing, solving the equation becomes significantly more straightforward by converting to bipolar coordinates. This transformation tailors the coordinate system to better fit the geometric complexities often encountered in non-circular cross-section scenarios, enhancing the mathematical handling and solution accuracy of the flow dynamics.

The axes ξ and η of the bipolar coordinate system and the axes x and y of the Cartesian coordinate system satisfy the relationship shown in mathematical Expression 2 below.

$$x = \frac{a sinh η}{cosh η - cos ξ} , y = \frac{a sin ξ}{cosh η - cos ξ} \qquad [ Expression 2 ]$$

where 2a is the inter-focal distance. Here ξ coordinate represents one of the two angular coordinates in bipolar coordinates. It measures the angle formed by the line connecting the point to one focus and the line perpendicular to the line connecting the two foci. Essentially, ξ can be thought of as describing the angles around each focus. On the other hand, η coordinate is the second angular coordinate and measures the logarithmic distance ratio of a point to the two foci. FIG. 2 illustrates the bipolar coordinates. The coordinate ξ varies from ξ_* on the upper wall of BAV to π and from π to 2π-ξ_* on the lower wall of BAV.

The steady state solution u0 of Expression 1 satisfies no-slip boundary condition as the following Expression 3

$${u}_{0} = ( {ξ}_{*} , η ) = 0 , {u}_{0} ( 2 π - {ξ}_{*} ) = 0 \qquad [ Expression 3 ]$$

Here, ξ* is the χ value corresponding to the valve edge.

Then the steady flow is given by the following Expression 4.

$${u}_{0} ( ξ , η ) = - \frac{{ka}^{2}}{2 μ} \frac{sin ( ξ - {ξ}_{* j} )}{sin ( {ξ}_{* j} ) ( cosh η - cos ξ )} , \qquad [ Expression 4 ]$$

with ξ_(*j)=ξ_* for ξ_*≤ξ≤π and ξ_(*j)=2π−ξ_* for π≤ξ≤2π−ξ_*. The steady flow reduces to that of circular cylindrical tube u_circular of equivalent diameter when ξ_*=π/2, ξ=π and η=0, given by the following Expression 5.

$${u}_{circular} ( x = 0 , y = 0 ) = - \frac{{ka}^{2}}{4 μ} \qquad [ Expression 5 ]$$

In addition, the blood flow Q obtained by integrating the above steady-state equation is expressed as in Equation 7 below.

$$Q = \frac{k}{4 μ} [ {a}^{4} cot {ξ}_{*} ( \frac{4}{3} + \frac{4}{3} {cot}^{2} {ξ}_{*} - 4 {csc}^{2} {ξ}_{*} ) + {a}^{4} {csc}^{4} {ξ}_{*} ( - {ξ}_{*} - \frac{2}{3} sin 2 {ξ}_{*} - \frac{1}{16} sin 4 {ξ}_{*} ) ] . \qquad [ Expression 6 ]$$

When ξ*=π/2, the blood flow Q becomes the flow in a circular cylindrical tube of equivalent diameter, and the following Expression 7 is derived.

$$Q = {Q}_{circular} = - \frac{k π {a}^{4}}{8 μ} \qquad [ Expression 7 ]$$

In addition, the shear stress τ(ξ, η) applied to the aorta obtained from the above steady-state equation is expressed as in Expression 8 below.

$$\begin{matrix}τ = - μ \frac{cosh η - cos ξ}{a} \frac{\partial u ( ξ , η )}{\partial ξ} \\ = \frac{ka}{2 sin {ξ}_{* j}} [ cos ( ξ - {ξ}_{* j} ) - \frac{sin ( ξ - {ξ}_{* j} ) sin ξ}{cosh η - cos ξ} ]\end{matrix} . \qquad [ Expression 8 ]$$

The wall shear stress (WSS) at the edge (ξ*j) of the bicuspid aortic valve is expressed as in the following Expression 9.

$${τ \vert}_{{ξ}_{* j}} = \frac{ka}{2 sin {ξ}_{* j}} \qquad [ Expression 9 ]$$

which can be compared with that of the circular cylindrical tube with the equivalent diameter as the following Expression 10.

$${τ}_{circular} = \frac{ka}{2} \qquad [ Expression 10 ]$$

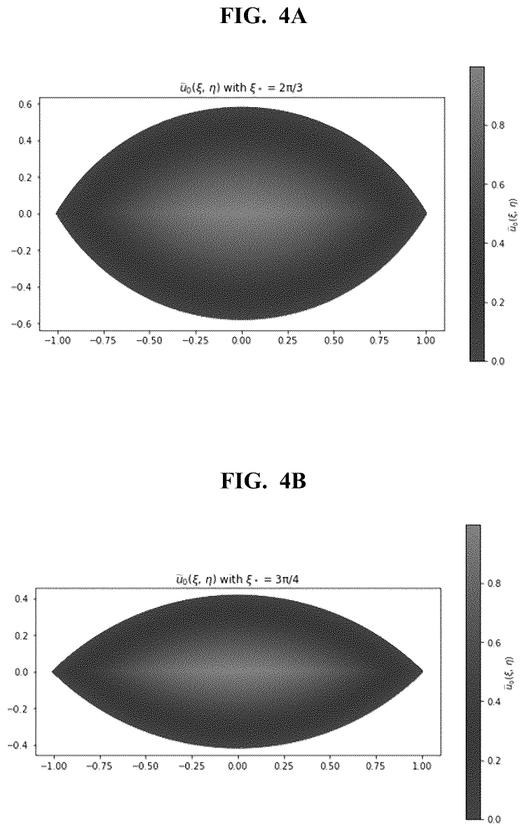

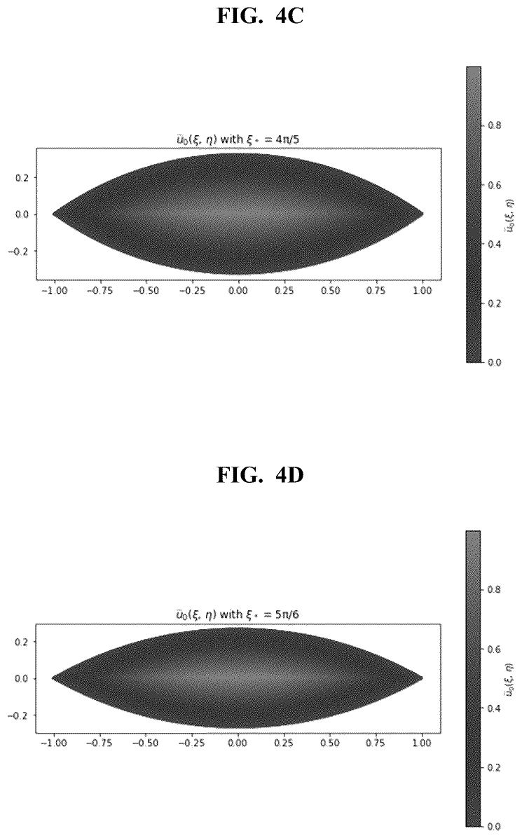

FIG. 4A illustrates the steady flow through the bipolar-shaped orifice, depicting the wall boundaries at positions ξ_{*}=2π/3, for bicuspid aortic valve, FIG. 4B illustrates the steady flow through the bipolar-shaped orifice, depicting the wall boundaries at positions ξ_{*}=3π/4, for bicuspid aortic valve, FIG. 4C illustrates the steady flow through the bipolar-shaped orifice, depicting the wall boundaries at positions ξ_{*}=4π/5, for bicuspid aortic valve, and FIG. 4D illustrates the steady flow through the bipolar-shaped orifice, depicting the wall boundaries at positions ξ_{*}=5π/6, for bicuspid aortic valve.

The velocity profile is normalized to the peak velocity observed in the TAV with an equivalent diameter. This normalization allows for a direct comparison between the flow characteristics of the BAV and TAV, ensuring that differences in their respective velocity profiles are highlighted relative to a common reference point. The velocity profiles for the BAV show a remarkable agreement with those obtained in previous studies using coherent multi-scale simulations. These profiles consistently demonstrate the presence of a jet-like flow structure within the fluid, a feature that is notably absent in the TAV scenarios. This jet formation is indicative of the distinct hemodynamic patterns associated with BAV, underscoring the significant impact of valve morphology on flow dynamics.

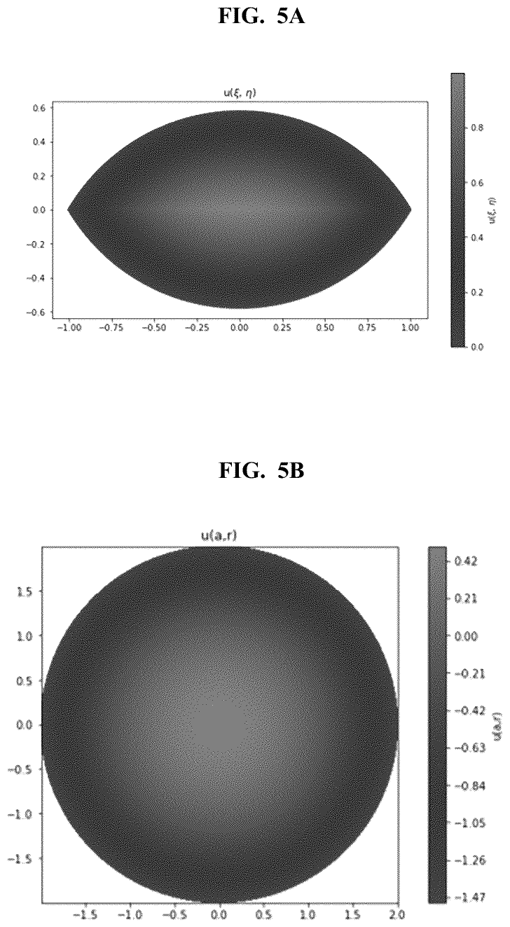

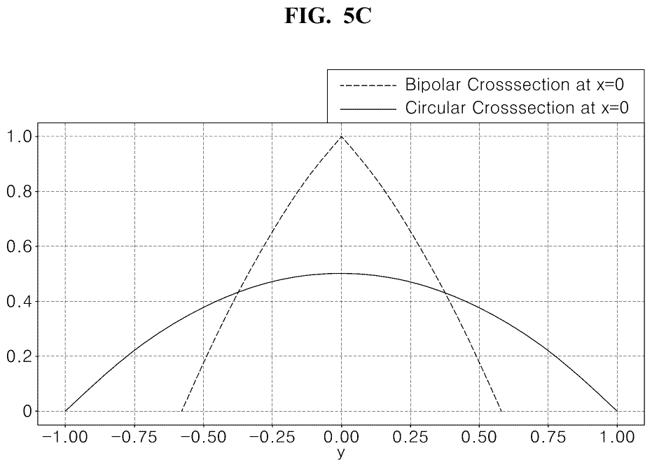

FIG. 5A presents comparative velocity profiles at the aorta entrance for BAV, FIG. 5B presents comparative velocity profiles at the aorta entrance for TAV, and FIG. 5C presents comparative velocity profiles at the aorta entrance for a combined profile showing BAV (blue) and TAV (red) at the center.

The analysis reveals that at the center of the entrance, the velocity for the bicuspid aortic valve (BAV) is significantly higher compared to the tricuspid aortic valve (TAV). However, the BAV velocity decreases more rapidly than that of the TAV as it moves towards the vessel wall. This rapid decrease in velocity for the BAV creates a steeper velocity gradient in the vertical direction towards the vessel wall. Consequently, this results in higher wall shear stress in the case of BAV. The increased wall shear stress can have significant implications for vascular health, potentially influencing the development of aortic diseases and complications associated with BAV. Our results demonstrate a reasonably good agreement with FIG. 7. The computation time was on the order of minutes for each cell, whereas our analytical model-based computation achieves similar results in just a few seconds. This significant reduction in computation time highlights the efficiency and effectiveness of our approach, providing rapid and reliable analysis that can be advantageous for both research and clinical applications.

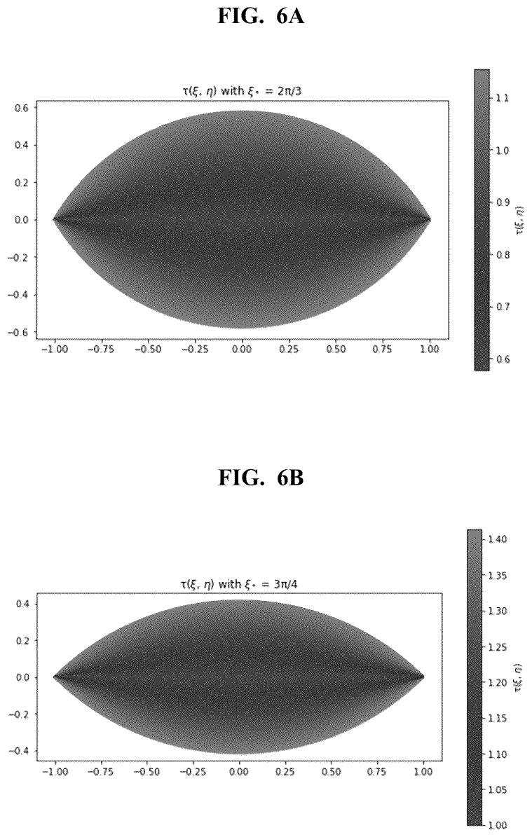

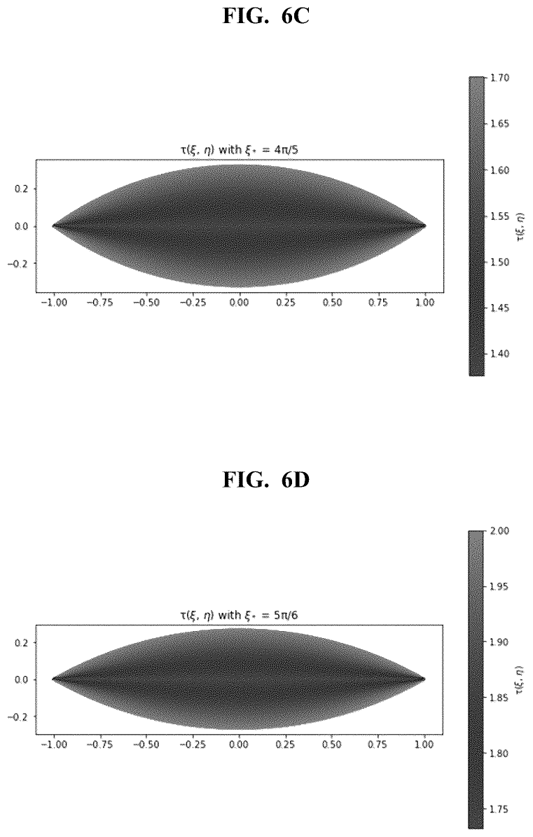

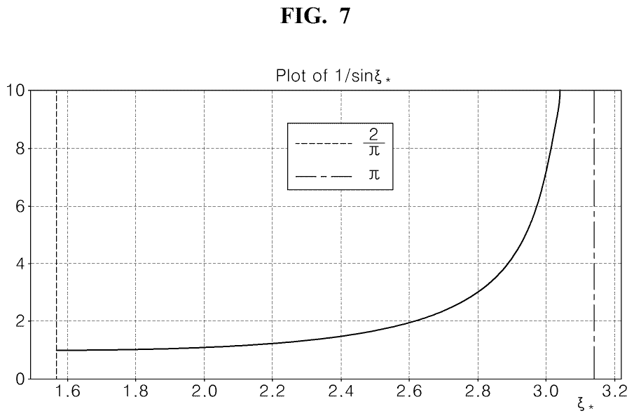

FIG. 6A illustrates the normalized shear stress distribution across the bipolar-shaped orifice, highlighting the wall boundaries at positions ξ_{*}=2π/3, FIG. 6B illustrates the normalized shear stress distribution across the bipolar-shaped orifice, highlighting the wall boundaries at positions ξ_{*}=3π/4, FIG. 6C illustrates the normalized shear stress distribution across the bipolar-shaped orifice, highlighting the wall boundaries at positions ξ_{*}=4π/5, and FIG. 6D illustrates the normalized shear stress distribution across the bipolar-shaped orifice, highlighting the wall boundaries at positions ξ_{*}=5π/6.

As derived from Expression 7 and discussed in the context of FIG. 5, the wall shear stress (WSS) reaches its maximum at the boundary of the bicuspid aortic valve. The analysis reveals that as the shape of the bicuspid valve becomes narrower, the WSS increases significantly. This indicates that the geometry of the bicuspid valve has a critical impact on the shear stress experienced at the vessel wall, with narrower valve shapes leading to higher shear stress. This finding is essential for understanding the hemodynamic stresses associated with bicuspid aortic valves and their potential implications for vascular health.

FIG. 7 illustrates WSS of BAV normalized by the WSS of TAV.

The normalized WSS is inversely proportion to sin(ξ_*) which is rapidly increasing as the orifice of the aortic valve becomes more asymmetrical.

In FIG. 7, we plot the wall shear stress (WSS) of the bicuspid aortic valve (BAV) normalized by the WSS of the tricuspid aortic valve (TAV). The normalized WSS is inversely proportional to sin(ξ_*) of the aortic valve orifice, rapidly increasing as the orifice becomes more asymmetrical. This demonstrates that as the aortic valve deviates from a symmetric shape, the WSS increases significantly, which can have important implications for the structural integrity and function of the valve.

Stable blood flow through compressed or defective vessels, i.e. Poiseuille flow, is an important problem in hemodynamics, particularly in cardiovascular research. This invention investigates a tube with a bipolar cross-section to simulate the elliptical systolic opening of a bicuspid aortic valve (BAV). Unlike the normal tricuspid aortic valve (TAV), the BAV, which has two leaflets, presents a unique hemodynamic problem. BAV, the most common congenital heart defect, is associated with an increased risk of aortic dilatation and dissection among patients.

Bipolar cross-section analysis provides a more accurate geometric approximation for modeling flow through this atypical valve shape and is important for understanding the specific fluid dynamics associated with BAV. In this invention, an accurate solution to the governing equations of Poiseuille flow in a tube with a bipolar cross-section is derived. The results include a detailed analysis of the velocity field, flow rate, and wall shear stress (WSS).

The results show that the velocity profile of the BAV is remarkably consistent with those obtained through previous multi-scale simulations. These profiles consistently exhibit jet-like flow structures within the fluid that are not observed in the TAV scenario. Analysis shows that the flow velocity in BAV is significantly higher at the inlet center than in TAV. However, the flow velocity in BAV decreases more rapidly toward the vessel wall, creating a steeper vertical velocity gradient. This results in higher wall shear stress in BAV. Furthermore, wall shear stress (WSS) is proportional to the inverse of sin(ξ_) as ξ represents the bipolar coordinate of the wall boundary, and is significantly higher than that found in a cylindrical tube of equivalent diameter. In the case of aortic valve stenosis where ξ approaches π, the WSS increases very sharply. This elevated WSS is commonly observed in patients with BAV, and may negatively affect the aortic wall, particularly in the ascending aorta, contributing to the higher incidence of aortic complications in these patients. Understanding these hemodynamic factors is essential for developing better diagnostic and therapeutic strategies for the management of BAV-related diseases.

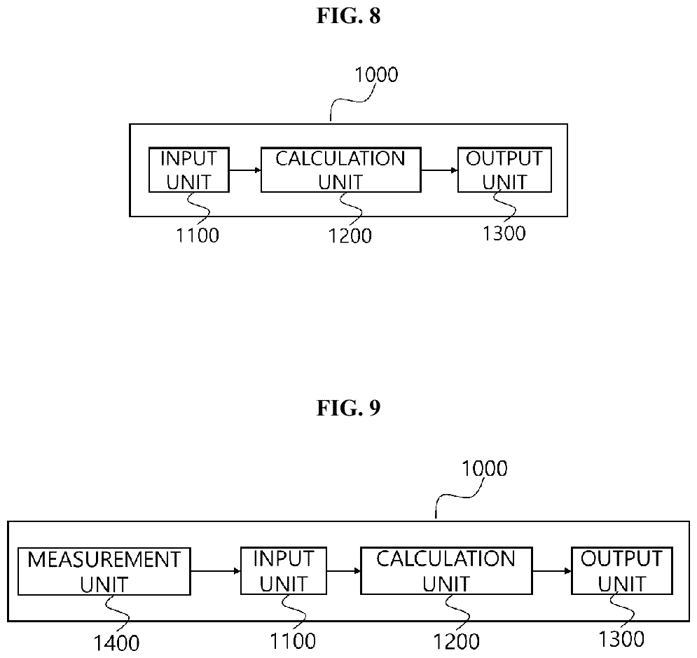

FIG. 8 is a block diagram illustrating a medical device according to an exemplary embodiment of the present invention.

Referring to FIG. 8, the medical device 1000 according to the present invention uses the above method of calculating blood flow of the aorta and shear stress of the aorta, and includes an input unit 1100, a calculation unit 1200, and an output unit 1300. The input unit 1100 receives the characteristic value of the bicuspid aortic valve. More specifically, as an example, the characteristic value may be input by a user. At this time, the input unit may be implemented through a keyboard, mouse, touch screen, etc. That is, the user may input the characteristic value obtained through equipment capable of projecting blood vessels, such as separate ultrasound, CT, MRI, etc., into the medical device 1000 according to the present invention through the above input unit 1100. The above calculation unit 1200 calculates the blood flow per unit area and the shear stress applied to the aorta in the bipolar coordinate system of the governing equation using the characteristic value received from the input unit 1100. Since the detailed calculation process has been described above, a duplicate description will be omitted. The output unit 1300 outputs the result calculated from the association unit. The output unit can be implemented as a device such as a display, a printer, etc.

FIG. 9 is a block diagram illustrating a medical device according to another exemplary embodiment of the present invention. The medical device illustrated in FIG. 9 is substantially the same as the medical device illustrated in FIG. 8 except that it further includes a measuring unit and the input unit receives characteristic values from the measuring unit. Therefore, identical or similar components are given the same reference numerals and duplicate descriptions are omitted.

Referring to FIG. 9, the characteristic values can be input from the measuring unit 1400. At this time, the measuring unit 1400 is equipment capable of projecting blood vessels, such as ultrasound, CT, MRI, etc., and the medical device 1000 according to the present invention is integrated with such conventional measuring devices, and can immediately calculate and display the blood flow per unit area and the shear stress applied to the aorta.

In addition, the electronic calculator-readable recording medium according to the present invention stores a program to perform the above method for calculating the blood flow of the aorta and the shear stress of the aorta. Such a recording medium can be implemented in the form of program commands that can be executed through various computer means and recorded on a computer-readable medium. The above computer-readable medium may include program commands, data files, data structures, etc., alone or in combination. The program commands recorded on the medium may be those specially designed and configured for the embodiment or may be those known and available to those skilled in the art. Examples of the computer-readable recording medium include magnetic media such as hard disks, floppy disks, and magnetic tapes, optical media such as CD-ROMs and DVDs, magneto-optical media such as floptical disks, and hardware devices specially configured to store and execute program commands such as ROMs, RAMs, flash memories, etc. Examples of the program commands include not only machine language codes generated by a compiler, but also high-level language codes that can be executed by a computer using an interpreter, etc. The above hardware devices may be configured to operate as one or more software modules to perform the operations of the embodiment, and vice versa. As described above, according to the method for calculating blood flow and shear stress of the aorta by the present invention, compared to the conventional method of using a simulation by the Navier-Stokes equation using the finite element method (F.E.M.), the bicuspid aortic valve is approximated by a bipolar coordinate system having a similar shape, and the directly obtained solution is used, thereby reducing the speed of calculation and enabling rapid diagnosis.

It will be apparent to those skilled in the art that various modifications and variation may be made in the present invention without departing from the spirit or scope of the invention. Thus, it is intended that the present invention cover the modifications and variations of this invention provided they come within the scope of the appended claims and their equivalents.

What is claimed is:

1. A method for calculating blood flow and shear stress of aorta having a bicuspid aortic valve, which is performed by an electronic device, the method comprising:
receiving characteristic values of a bicuspid aortic valve; and
calculating blood flow per unit area and shear stress applied to the aorta in a bipolar coordinate system of governing equation.

2. The method of claim 1, wherein the characteristic values of a bicuspid aortic valve are interpole distance (2a) in the bipolar coordinate system and the corresponding ξ_* value at the valve edge.

3. The method of claim 1, wherein the governing equation is expressed as
$$\frac{\partial P}{\partial z} = μ ( \frac{{\partial }^{2} u}{\partial {x}^{2}} + \frac{{\partial }^{2} u}{\partial {y}^{2}} )$$
wherein P is the pressure, u(x,y) is the velocity in the z-direction, μ is the viscosity of the blood, and x and y represent each axis of the Cartesian coordinate system, respectively.

4. The method of claim 3, wherein a solution of the governing equation in the bipolar coordinate system is obtained by a boundary condition expressed as
$${u}_{0} ( {ξ}_{*} , η ) = {u}_{0} ( 2 π - {ξ}_{*} ) = 0.$$

5. The method of claim 4, wherein a steady state solution obtained by the boundary condition is expressed as
$${u}_{0} ( ξ , η ) = - \frac{{ka}^{2}}{2 μ} \frac{sin ( ξ - {ξ}_{* j} )}{sin ( {ξ}_{* j} ) ( cosh η - cos ξ )} ,$$
$$wherein$$
$${ξ}_{* j} = {ξ}_{*} for {ξ}_{*} \leq ξ \leq π and {ξ}_{* j} = 2 π - {ξ}_{*} for π \leq ξ \leq 2 π - {ξ}_{*} .$$

6. The method of claim 5, wherein a solution involving time t due to the contraction of the heart is approximated by multiplying the steady-state solution by the value of e^{iωt}.

7. The method of claim 5, wherein a blood flow Q obtained by integrating the steady-state equation is expressed as
$$Q = \frac{k}{4 μ} [ {a}^{4} cot {ξ}_{*} ( \frac{4}{3} + \frac{4}{3} {cot}^{2} {ξ}_{*} - 4 {csc}^{2} {ξ}_{*} ) + {a}^{4} {csc}^{4} {ξ}_{*} ( - {ξ}_{*} - \frac{2}{3} sin 2 {ξ}_{*} - \frac{1}{16} sin 4 {ξ}_{*} ) ] .$$

8. The method of claim 5, wherein a shear stress τ(ξ,η) applied to the aorta obtained from the above steady-state equation is expressed as
$$\begin{matrix}τ = - μ \frac{cosh η - cos ξ}{a} \frac{\partial u ( ξ , η )}{\partial ξ} \\ = \frac{ka}{2 sin {ξ}_{* j}} [ cos ( ξ - {ξ}_{* j} ) - \frac{sin ( ξ - {ξ}_{* j} ) sin ξ}{cosh η - cos ξ} ]\end{matrix} .$$

9. The method of claim 8, wherein a shear stress at the end (ξ*j) of a bicuspid aortic valve is expressed as
$${τ \vert}_{{ξ}_{* j}} = \frac{ka}{2 sin {ξ}_{* j}}$$

10. A computer-readable recording medium for performing the method for calculating blood flow and shear stress of an aorta having a bicuspid aortic valve according to claim 1.

11. A medical device using the method for calculating blood flow and shear stress of an aorta having a bicuspid aortic valve according to claim 1, comprising:
an input unit for receiving characteristic values of a bicuspid aortic valve;
a calculation unit for calculating blood flow per unit area and shear stress applied to an aorta in a bipolar coordinate system of a governing equation using the characteristic values received from the input unit; and
an output unit for outputting the result calculated from the association unit.

12. The medical device of claim 11, wherein the characteristic values are received from a user.

13. The medical device of claim 11, wherein the characteristic values are received from a measurement unit.
